(19) United States
(12) Patent Application Publication
(10) Pub. No.: US 2008/0300641 A1
(43) Pub. Date: Dec. 4, 2008

(54) CARDIAC INFORMATION AND ACTIVITY INFORMATION ASSOCIATION SYSTEMS, APPARATUS, AND METHODS

(76) Inventors: Willie N. Brunekreeft, Dieren (NL); Joanneke G. Groen, Velp (NL); Christianus J.J.E. Van Groeningen, Utrecht (NL)

Correspondence Address: MEDTRONIC, INC., 710 MEDTRONIC PARKWAY NE, MINNEAPOLIS, MN, 55432-9924, US

(21) Appl. No.: 11/756,683

(22) Filed: Jun. 1, 2007

(51) Int. Cl. A61N 1/39

(52) U.S. Cl. 607/6; 607/19

(57) ABSTRACT

Embodiments include cardiac information and activity information association systems, apparatus, and methods. An apparatus embodiment includes a cardiac sensor adapted to generate cardiac information descriptive of cardiac functioning of a patient, an activity sensor adapted to generate activity information indicating physical activity of the patient, a processing element adapted to detect a cardiac anomaly based on the cardiac information, an information association element adapted to generate associated cardiac/activity information during the cardiac anomaly, and a data storage apparatus adapted to store the associated cardiac/activity information. A method embodiment includes generating cardiac information descriptive of cardiac functioning of a patient, detecting a cardiac anomaly based on the cardiac information, generating activity information descriptive of physical activity of the patient during the cardiac anomaly, associating the cardiac information and the activity information, during the cardiac anomaly, to generate associated cardiac/activity information, and storing the associated cardiac/activity information.

### TECHNICAL FIELD

Embodiments of the inventive subject matter relate to systems, apparatus, and methods adapted to associate cardiac information and activity information.

### BACKGROUND

In an adult, a typical resting heart rate range is between about 60 beats per minute and about 100 beats per minute. Cardiac arrhythmia refers to a group of conditions in which the heartbeat is irregular or is faster or slower than normal. “Tachycardia” refers to an arrhythmia associated with a resting heart rate in excess of about 100 beats per minute. “Bradycardia” refers to an arrhythmia associated with a resting heart rate lower than about 60 beats per minute. Finally, “fibrillation” refers to a very serious variety of arrhythmia that occurs when the myocardium quivers due to disunities in contractile cell function. Fibrillation can be atrial or ventricular, primarily affecting the atria or the ventricles, respectively.

An arrhythmia that occurs due to increased activity level or stress typically is referred to as a “sympathetic” arrhythmia. In contrast, an arrhythmia that occurs at night or during periods of rest is typically referred to as a “vagal” arrhythmia. Some arrhythmias are common and of little concern, such as sympathetic arrhythmias that naturally occur when the sinoatrial node increases its rate of electrical activity to accelerate the heartbeat during exercise. Other arrhythmias may be abnormal, and yet may only cause annoying symptoms, such as palpitations, the awareness of an irregular heartbeat or may indicate serious underlying medical conditions for which medical evaluation and treatment should be sought.

Various treatments are commonly used to provide therapy to patients who experience recurrent arrhythmias. The type of treatment administered depends on the type of arrhythmia, when the arrhythmia occurs, and the underlying physical condition that is causing the arrhythmia to occur, if it can be diagnosed. For example, the type of treatment administered depends on whether the arrhythmia is bradycardia, tachycardia or fibrillation, whether the arrhythmia is atrial or ventricular, and whether the arrhythmia is sympathetic or vagal. A pacemaker is a common therapy for patients experiencing bradycardia, for example. Ablation and drug treatment are other options, which may be provided to patients experiencing atrial fibrillation or ventricular tachycardia.

In order to determine which treatment (or combination of treatments) may be most effective, data regarding a patient's cardiac health and functioning is first collected. An arrhythmia initially may be detected by auscultation of the heartbeat with a stethoscope. However, this method is non-specific, and an arrhythmia may not occur while auscultation is being performed, since arrhythmias typically occur spontaneously and unpredictably. An electrocardiogram (ECG) may be used to provide more specific information regarding the heart rhythm, and thus may be a more effective diagnostic tool. To capture ECG information at the onset of and during a spontaneous arrhythmia, a patient may wear an ambulatory electrocardiography device (e.g., a “Holter monitor”), which may store ECG data over a period of time (e.g., a 24-hour period).

Once ECG data has been collected, the information may be downloaded to a computer, and an experienced reviewer may manually analyze the information to identify heart rate variabilities that indicate episodes of arrhythmia. The reviewer also may correlate the data with other information to try to determine any precipitating factors behind arrhythmias indicated in the data. For example, a patient may keep a written log that indicates his or her levels of physical activity and/or stress during the ECG data-gathering period. The reviewer may correlate the physical activity log with identified episodes of arrhythmia to facilitate a determination of whether the arrhythmias occurred during rest or exercise, and to determine whether the arrhythmia occurred at night or during the day (e.g., whether the arrhythmia is sympathetic or vagal). These determinations may help medical personnel to apply effective therapies.

Current methods for collecting and correlating ECG data with physical activity information to determine precipitating factors behind arrhythmias or other cardiac anomalies (e.g., myocardial ischemia, among other things) suffer from some drawbacks. For example, patients may not be diligent or accurate in recording information regarding physical activity or stress levels, and patient-recorded times may not be accurately synchronized with ECG time marker information. These inaccuracies may make diagnosis more difficult and inaccurate, and may further lead to determinations of non-optimal treatment strategies. For at least the foregoing reasons, a need exists for systems, apparatus, and methods adapted to accurately collect and correlate cardiac data and physical activity information to facilitate accurate diagnosis of the causes behind recurring arrhythmias or other cardiac anomalies and to facilitate determination of effective treatment strategies.

### BRIEF SUMMARY

A method embodiment of the inventive subject matter includes generating cardiac information descriptive of cardiac functioning of a patient, detecting a cardiac anomaly based on an analysis of the cardiac information, generating activity information descriptive of physical activity of the patient during the cardiac anomaly, associating the cardiac information and the activity information, during the cardiac anomaly, to generate associated cardiac/activity information, and storing the associated cardiac/activity information.

An apparatus embodiment of the inventive subject matter includes at least one cardiac sensor adapted to generate cardiac information descriptive of cardiac functioning of the patient, at least one activity sensor adapted to generate activity information indicating physical activity of the patient, a processing element adapted to receive the cardiac information and to detect a cardiac anomaly based on an analysis of the cardiac information, an information association element adapted to generate associated cardiac/activity information during the cardiac anomaly, and at least one data storage apparatus adapted to store the associated cardiac/activity information.

A system embodiment of the inventive subject matter includes a medical diagnostic system having an implantable cardiac apparatus having at least one cardiac sensor adapted to generate cardiac information descriptive of cardiac functioning of a patient, at least one activity sensor adapted to generate activity information indicating physical activity of the patient, a processing element adapted to receive the cardiac information and to detect a cardiac anomaly based on an analysis of the cardiac information, an information association element adapted to generate associated cardiac/activity information during the cardiac anomaly, at least one data storage apparatus adapted to store the associated cardiac/activity information, and at least one output device adapted to output the associated cardiac/activity information. In an embodiment, the medical diagnostic system also includes an external apparatus with a receiver adapted to receive the associated cardiac/activity information, and a display device adapted to display the associated cardiac/activity information.

### BRIEF DESCRIPTION OF THE DRAWINGS

The inventive subject matter will be described in conjunction with the following drawing figures, in which:

FIG. 1 illustrates the interior anatomy of a human heart;

FIG. 2 illustrates a normal ECG waveform and an ECG waveform that is characteristic of atrial fibrillation;

FIG. 3 illustrates a perspective view of a cardiac/activity information association system, in accordance with an example embodiment of the inventive subject matter;

FIG. 4 illustrates a functional block diagram of a cardiac/activity information association system, in accordance with an example embodiment; and

FIG. 5 illustrates a flowchart of a method for collecting and associating cardiac information and activity information, in accordance with an example embodiment.

### DETAILED DESCRIPTION

The following detailed description of the inventive subject matter is merely exemplary in nature and is not intended to limit the inventive subject matter or the application and uses of the inventive subject matter. Furthermore, there is no intention to be bound by any theory presented in the preceding background or the following detailed description.

Embodiments of the inventive subject matter include systems, apparatus, and methods adapted to collect and associate cardiac information with activity information. These systems, apparatus, and methods may be referred to herein as “cardiac/activity information association” systems, apparatus, and methods, for purposes of brevity. In a particular embodiment, which will be described in detail later, a cardiac/activity information association system may include one or more cardiac data sensing and analysis apparatus, one or more activity data sensing and analysis apparatus, and a cardiac and activity information association apparatus.

In various embodiments, collected and/or analyzed cardiac information may indicate the presence of a cardiac anomaly. As used herein, the term cardiac anomaly includes arrhythmias (e.g., bradycardia, tachycardia, and fibrillation), myocardial ischemia, myocardial infarction, and any other cardiac event that deviates from normal cardiac functioning. Example embodiments described below discuss systems, apparatus, and methods adapted to collect and associate cardiac and activity information in the context of detecting arrhythmias. It is to be understood that embodiments of the inventive subject matter are not limited to use in the context of arrhythmias, but may be adapted to be used in the context of other cardiac anomalies, as well. In order to describe embodiments of the inventive subject matter clearly, a description of a human heart and its functioning is included below.

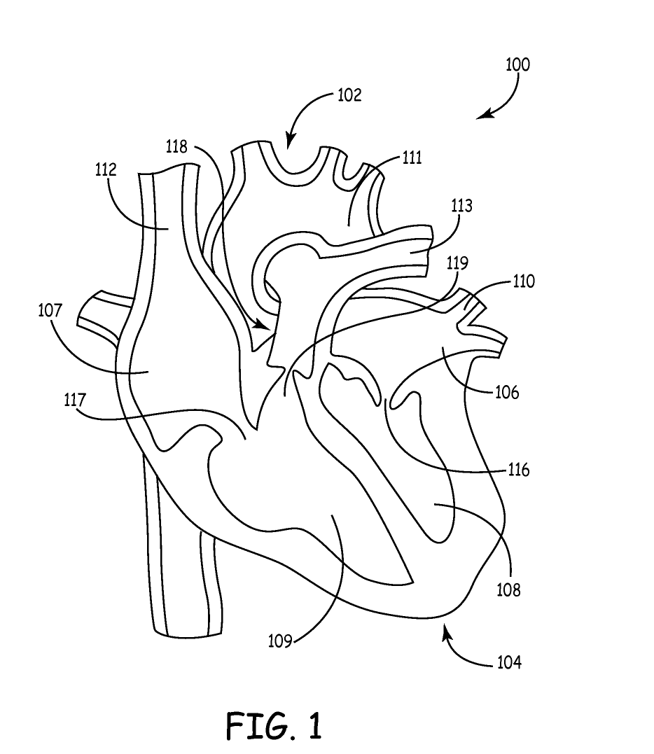

FIG. 1 illustrates the interior anatomy of a human heart 100. The top of the heart 100 may be referred to as the base 102, and the bottom of the heart may be referred to as the apex 104. The heart 100 includes four chambers: a left atrium 106, a right atrium 107, a left ventricle 108, and a right ventricle 109. During a cardiac cycle, the heart chambers 106-109 contract and relax in response to electrical currents periodically conveyed by a biological conduction system (not illustrated).

During a portion of the cardiac cycle referred to as diastole, the left atrium 106 relaxes and fills with blood from the lungs via the upper pulmonary vein 110 (i.e., pulmonary veins from the left and right lungs). The right atrium 107 also relaxes during diastole, and fills with blood from the body via the superior vena cava 112 and the inferior vena cava (not shown in FIG. 1). The blood within the left atrium 106 enters the left ventricle 108 through the mitral valve 116. The left ventricle 108 subsequently pumps the blood through the aortic valve 118 (hidden in FIG. 1) and into the body via the aorta 111 during a phase of the cardiac cycle referred to as systole.

During a portion of the cardiac cycle referred to as diastole, the blood within the right atrium 107 enters the right ventricle 109 through the tricuspid valve 117. The right ventricle 109 subsequently pumps the blood through the pulmonary valve 119 and into the lungs via the pulmonary artery 113 during systole.

The set of four heart valves function to regulate blood flow through the chambers 106-109 of the heart 100, by opening and closing at various times. These valves include the atrioventricular (AV) valves 116, 117 and the semilunar (SL) valves 118, 119. The AV valves include the mitral valve 116 and the tricuspid valve 117. The AV valves 116, 117 open during diastole to allow the ventricles to fill with blood. The SL valves 118, 119, which are set between the arteries 106, 107 and the ventricles 108, 109, include the aortic valve 118 and the pulmonary valve 119. The SL valves 118, 119 open during systole to allow blood to be ejected from the heart 100.

During a cardiac cycle (i.e., a heartbeat), a sequence of events occurs within the heart 100, as described above. As mentioned previously, each cardiac cycle includes a diastole portion and a systole portion. More specifically, each cardiac cycle includes three major stages: atrial systole; ventricular systole; and complete cardiac diastole. These three stages may be monitored by an ECG, which senses the pattern of electrical impulses generated at the heart during the stages of the cardiac cycle.

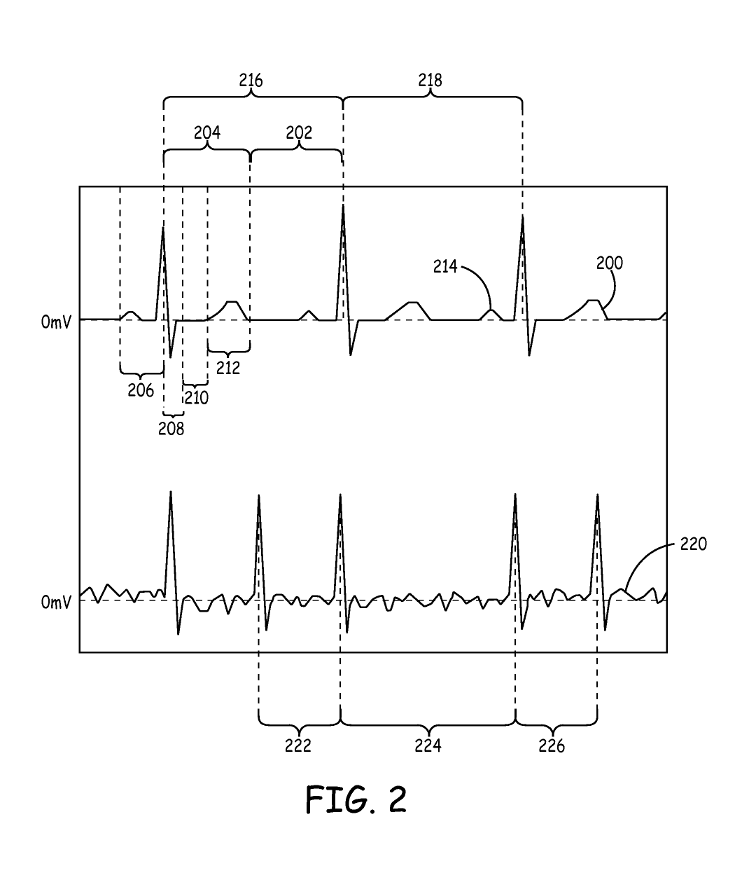

FIG. 2 illustrates a normal ECG waveform 200 and an ECG waveform 220 that is characteristic of atrial fibrillation. Referring first to ECG waveform 200, a normal cardiac cycle includes TR segment 202 and an RT segment 204. The TR segment 202 includes a P wave 206. The P wave 206 indicates the movement of electrical activity through the upper heart chambers. Atrial systole occurs at the onset of the P wave 206, and indicates the contraction of the myocardium (i.e., heart muscle tissue) of the left and right atria. Atrial systole includes an electrical systole (i.e., the electrical activity that stimulates contraction of the myocardium of the heart chambers) followed by a mechanical systole (i.e., the mechanical contraction of the heart chambers).

Complete cardiac diastole occurs during the TR segment 202, and indicates the relaxation of the heart muscle after contraction in preparation for refilling with circulating blood. Complete cardiac diastole includes a ventricular diastole period when the ventricles are relaxing, and an atrial diastole period when the atria are relaxing. During the ventricular diastole period, the blood pressure in the left and right ventricles drops from the peak that it reached in systole. When the pressure in the left ventricle drops to a pressure below the pressure in the left atrium, the mitral valve opens, and the left ventricle fills with the blood that was accumulating in the left atrium. Likewise, when the pressure in the right ventricle drops below the pressure in the right atrium, the tricuspid valve opens, and the right ventricle fills with the blood that was accumulating in the right atrium.

The RT segment 204 of a cardiac cycle includes a QRS complex 208, ST segment 210, and T wave 212. The ST segment 210 normally appears as a straight, level line between the QRS complex 208 and the T wave 212. The T wave 212 corresponds to the period when the lower heart chambers are relaxing electrically and preparing for their next muscle contraction.

Ventricular systole occurs during RT segment 204, and indicates the contraction of the myocardium of the left and right ventricles. Similar to atrial systole, ventricular systole also includes an electrical systole followed by a mechanical systole. In an ECG 200, electrical systole of the ventricles begins at the beginning of the QRS complex 208 (i.e., a measurement of the movement of electrical impulses through the lower heart chambers).

A heart rate typically is measured as a number of ventricular contractions (i.e., heartbeats) per minute (or other time period), and heart rate variability indicates the variation over time of the heart rate. As indicated by ECG waveform 200, the time durations between consecutive heartbeats normally is approximately equal, as indicated by approximately equal durations 216, 218. During a respiration cycle and/or as activity levels increase or decrease, the durations between heartbeats may change, although the change normally is relatively smooth. During an episode of atrial fibrillation, such as is indicated by ECG waveform 220, the durations between consecutive heartbeats may vary erratically, as indicated by unequal durations 222, 224, 226.

ECG waveforms may indicate other cardiac anomalies, as well. For example, tachycardia may be indicated by a heart rate that exceeds a threshold (e.g., a resting heart rate of 100 beats/minute) and bradycardia may be indicated by a heart rate that is less than a threshold (e.g., a resting heart rate of 60 beats/minute). In addition, myocardial ischemia and/or cell necrosis may be indicated by an elevated or depressed ST segment and/or by an inverted T wave, for example.

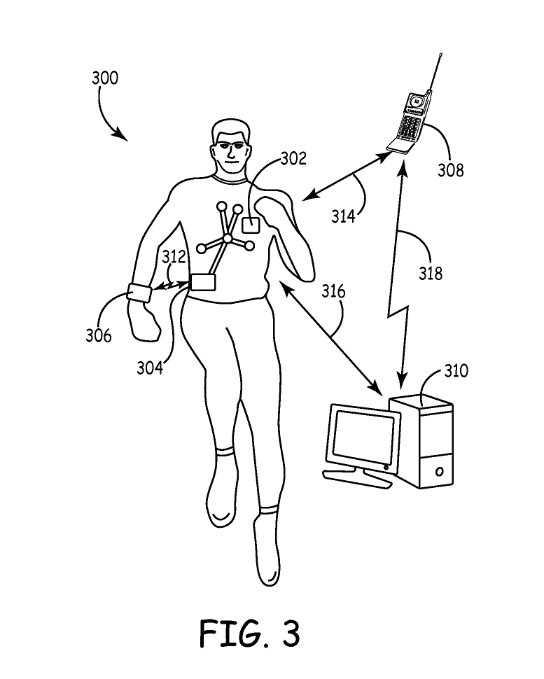

FIG. 3 illustrates a perspective view of a cardiac/activity information association system 300, in accordance with an example embodiment of the inventive subject matter. In an embodiment, system 300 may be a medical diagnostic system adapted to facilitate diagnosis of a cardiac condition. System 300 may include at least one host system 302, at least one cardiac sensing apparatus 304, and at least one activity sensing apparatus 306. Host system 302, cardiac sensing apparatus 304, and activity sensing apparatus 306 may be separate apparatus (e.g., separately packaged), as illustrated in FIG. 3, or any two or more of apparatus 302, 304, 306 may be incorporated together in a single package or device. For example, in a particular embodiment, cardiac sensing apparatus 304 and activity sensing apparatus 306 may form portions of host system 302. In various embodiments, host system 302, cardiac sensing apparatus 304, and/or activity sensing apparatus 306 may be implanted within and/or positioned externally on a patient. For ease of description and illustration, host system 302, cardiac sensing apparatus 304, and activity sensing apparatus 306 are shown as separate apparatus.

Host system 302 may be, for example, an apparatus adapted to be implanted in a patient. In addition, host system 302 may be a device adapted to sense cardiac functioning and/or to provide stimuli to the heart tissue. Host system 302 may include, for example but not by way of limitation, an implantable pulse generator (IPG) (e.g., a “pacemaker”), an implantable cardiac resynchronization therapy (CRT) device, an implantable cardioverter defibrillator (ICD), an implantable cardiac diagnostic and monitoring device, a unit that combines two or more of the aforementioned devices, or another device. Accordingly, host system 302 may include one or more stimulus elements (e.g., electrical and/or mechanical stimulus elements) adapted to be implanted within a patient. The stimulus elements may be coupled to host system 302 via leads, or they may have leadless (e.g., direct, wireless radio frequency, and/or ultrasound) connections to host system 302 and/or to each other. These leads may include transvenous leads and/or subcutaneous catheters, for example.

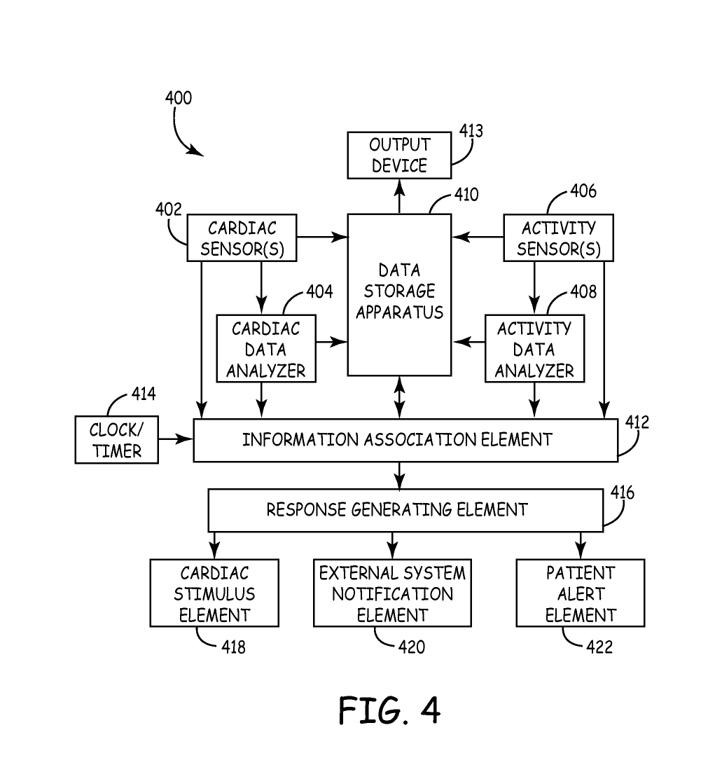

Cardiac sensing apparatus 304 may be a device adapted to sense and collect one or more types of cardiac data during an observation period (e.g., a 24-hour period). For example, but not by way of limitation, cardiac sensing apparatus 304 may include an electrocardiography device (e.g., a “Holter monitor”), which may store ECG data during the observation period. As will be described in more detail in conjunction with FIG. 4, cardiac sensing apparatus 304 may be further adapted to analyze cardiac data in order to calculate one or more cardiac function values that are descriptive of the patient's cardiac functioning during the observation period. The term “cardiac information,” as used herein, means cardiac data and/or cardiac function values. In various embodiments, cardiac information represents the cardiac data and/or cardiac function values.

Activity sensing apparatus 306 may be a device adapted to sense and collect data regarding physical activity of the patient during the observation period. In various embodiments, activity sensing apparatus 306 may be adapted to sense and collect various types of physical activities, including but not limited to activities in a group of activities that include limb movement, postures (e.g., lying, reclining, sitting, standing, leaning, etc.), posture transitions, gaits (e.g., jumping, walking, running, going up or down stairs, biking, etc.), and/or eating/drinking. As will be described in more detail in conjunction with FIG. 4, activity sensing apparatus 306 may be further adapted to analyze activity data to generate one or more activity level values that are indicative of a patient's activity levels. The term “activity information,” as used herein, means activity data and/or activity level values. In various embodiments, activity information represents the activity data and/or the activity level values.

In additional embodiments, system 300 may include one or more external apparatus, such as one or more communication devices 308 (e.g., a radio, wristwatch, telephone, pager, patient activator) and/or computing devices 310 (e.g., a computer). The external apparatus may include, for example, a receiver adapted to receive the cardiac information, the activity information, and/or the associated cardiac/activity information. In addition, the external apparatus may include a display device adapted to display the cardiac information, the activity information, and/or the associated cardiac/activity information. Any one or more of host system 302, cardiac sensing apparatus 304, activity sensing apparatus 306, patient communication device 308, and/or computing device 310 may be adapted to communicate with each other over one or more wired or wireless communication links, such as wireless links 312, 314, 316, 318 or other links (not illustrated). Various data may be exchanged over the communication links, including but not limited to cardiac information and/or activity information, among other things.

Communication device 308 and/or computing device 310 may connect over an external system to convey information regarding the patient to a remote individual or system. For example, communication device 308 and/or computing device 310 may connect over a cellular, radio, telephone, or computer network (e.g., the Internet) with a doctor's office, emergency response system (e.g., a 911 system), hospital, caretaker or other entity to convey information regarding the patient's cardiac information. The remote individual or system may be able to communicate with the host system 302, cardiac sensing apparatus 304, activity sensing apparatus 306, and/or the patient via patient communication device 308 and/or computing device 310 in order to send instructions (e.g., cardiac stimulus commands and/or patient instructions) and/or to obtain cardiac information and/or activity information, in an embodiment.

As will be described in more detail in conjunction with FIG. 4, system 300 may associate and store the cardiac information with the activity information, in various embodiments. The cardiac information and the activity information may correspond to cardiac data and activity data, respectively, sensed during an observation period. As used herein, the term “observation period” means a period of time during which cardiac data and activity data are sensed in order to evaluate the patient's cardiac functioning. An observation period may be very short (e.g., on the order of seconds) or may be extensive (e.g., on the order of hours or days). As will be described below, cardiac data and activity data are sensed during an observation period, and discrete segments of the cardiac data and activity data may be analyzed to generate cardiac information descriptive of the patient's cardiac functioning and activity information descriptive of the patient's physical activity, during the observation period. The discrete segments of the cardiac data and the activity data correspond to portions of the observation period and/or discrete times within the observation period. In various embodiments, a discrete segment may correspond to a particular heartbeat, a set of consecutive heartbeats, a time instant, and/or a time period (e.g., having a start time, a duration, and/or a stop time). Temporal values (e.g., times and durations) may be measured and/or quantified in the system based on an absolute clock, a system clock, and/or a counter.

FIG. 4 illustrates a functional block diagram of a cardiac/activity information association system 400, in accordance with an example embodiment. In an embodiment, system 400 may be included within an implantable cardiac apparatus. System 400 includes at least one cardiac sensor 402, at least one cardiac data analyzer 404, at least one activity sensor 406, at least one activity data analyzer 408, at least one data storage apparatus 410, and at least one information association element 412, in an embodiment. The at least one cardiac sensor 402 and the at least one cardiac data analyzer 404 may be included in a cardiac sensing apparatus (e.g., cardiac sensing apparatus 304, FIG. 3). Similarly, the at least one activity sensor 406 and the at least one activity data analyzer may be included in an activity sensing apparatus (e.g., activity sensing apparatus 306, FIG. 3). System 400 may also include one or more data output devices 413, a clock/timer component 414, a response generating element 416, a cardiac stimulus element 418, an external apparatus notification element 420, and/or a patient alert element 422, in various embodiments. In various embodiments, the sensors, analyzers, and elements of system 400 may be implemented on general purpose or special purpose hardware (e.g., one or more microprocessors, integrated circuits, and/or other hardware), and portions of the processes performed may be implemented in software that is executed on the system hardware. System 400 may be implemented in one or more compact and portable apparatus, in an embodiment, such as one or more battery-powered apparatus that may be carried or otherwise worn by a patient. In other embodiments, all or portions of system 400 may be implemented in non-portable apparatus, such as one or more computers or other electronic systems or apparatus.

The at least one cardiac sensor 402 may include any one or more sensors selected from a group of sensors that include, but are not limited to ECG sensors, heart sound sensors, heart rate sensors, heart rate variability sensors, heart wall motion sensors, and/or other sensors adapted to sense and collect cardiac data. As used herein, the term “cardiac data” includes, but is not limited to ECG data, heart sound data, and heart wall motion data.

Cardiac sensors 402 may include one or more sensing elements (not illustrated) adapted to be implanted within or positioned externally to a patient. The sensing elements may be coupled to system 400 via leads (e.g., transvenous leads and/or subcutaneous tethers), or they may have leadless (e.g., direct, wireless radio frequency, and/or ultrasound) connections to system 400 and/or to each other. The cardiac data generated by cardiac sensor 402 may be stored in a data storage apparatus 410 and/or provided to cardiac data analyzer 404 and/or information association element 412, in various embodiments.

Cardiac data analyzer 404 may include one or more functions, implemented in software and executed on a general purpose or special purpose processor, which analyze the cardiac data generated by cardiac sensor 402, and which generate cardiac function values. Cardiac function values may include, for example, values describing heart rate, heart rate variability, atrial/ventricle synchronicity, atrial rhythm, and/or ventricle rhythm. In an embodiment, the cardiac function values may include values specifically related to heart rate variability including, but not limited to, any one or more values selected from a group of values that includes RR interval, SDNN, SDANN, rMSSD, pNN50, HF (ms2 and/or NU), Ln HF, LF (ms2 and/or NU), Ln LF, and/or LF/HF. The cardiac function values generated by cardiac data analyzer 404 may be stored in a data storage apparatus 410 and/or provided to information association element 412, in various embodiments. In an embodiment, cardiac data analyzer 404 may further be adapted to analyze cardiac data and/or cardiac function values to determine whether they indicate the onset or presence of a cardiac anomaly (e.g., arrhythmia, ischemia, myocardial infarction, etc.). Upon detection of the onset or presence of a cardiac anomaly, cardiac data analyzer 404 may initiate storage of a marker, and/or may initiate a response, as will be described later.

The at least one activity sensor 406 may include any one or more sensors selected from a group of sensors that includes, but is not limited to, movement sensors (e.g., single-axis and/or multiple-axis accelerometers), body segment angle sensors, strain gauges, pedometers, and/or other sensors adapted to sense and collect activity information. The term “activity data” includes, but is not limited to acceleration data, posture-related data, location data (e.g., global positioning data), pressure or strain data, activity counts (e.g., steps or repetitions), and/or other types of activity data. The activity data generated by activity sensor 406 may be stored in a data storage apparatus 410 and/or provided to activity data analyzer 408 and/or information association element 412, in various embodiments.

Activity data analyzer 408 may include one or more functions, implemented in software and executed on a general purpose or special purpose processor, which analyze the activity data generated by activity sensor 406, and which generate activity level values. Activity level values may include, for example but not by way of limitation, instantaneous values, total values, and/or average values over a period of time, describing power output, activity speed, activity type, counts (e.g., steps or repetitions), distance, and/or energy expenditure (e.g., caloric consumption). The activity level values generated by activity data analyzer 408 may be stored in a data storage apparatus 410 and/or provided to information association element 412, in various embodiments.

Data storage apparatus 410 may include one or multiple data storage devices within one or more system apparatus (e.g., host system 302, cardiac sensing apparatus 304, activity sensing apparatus 306, communication device 308 and/or computing device 310, FIG. 3). In various embodiments, data storage apparatus 410 may include random access memory (RAM), read only memory (ROM) (e.g., programmable ROM), flash memory, or other memory elements. Data may be stored compressed or uncompressed, in various embodiments. Upon demand, data may be retrieved through a wired (e.g., a serial port or USB port) or wireless connection, in various embodiments.

Information association element 412 is adapted to associate cardiac information and activity information, in an embodiment, to generate “associated cardiac/activity information.” As used herein, the term “associate” means to associate together based on a common parameter, where a common parameter may include a common temporal parameter (e.g., a time instant, a start time, a stop time, a time period, a time duration and/or a counter value), a common event (e.g., a particular heartbeat, set of consecutive heartbeats, cardiac anomaly, and/or a patient input), and/or a common quality (e.g., whether a cardiac anomaly is indicated by the information). In a particular embodiment, information association element 412 is adapted to associate cardiac information and activity information based on common temporal parameters (e.g., the cardiac information and the activity information correspond to data sensed at approximately a same time instant or within a same time period). Accordingly, in an embodiment, information association element 412 is adapted to associate cardiac information and activity information based on temporal information, in an embodiment. As used herein, the term “temporal information” includes time stamps (e.g., discrete time indicators, start times, and/or stop times), time period indicators (e.g., 24-hour period, 1 hour period, 5 minute period, etc.), counter values, and/or other indicia that indicates when, during a data collection period, cardiac or activity data was sensed. In an embodiment, system 400 includes at least one clock and/or timer component 414, which enables information association element 412 to determine temporal information. In other embodiments, cardiac information and activity information may be associated based on other common parameters (e.g., common events and/or common qualities, as discussed above).

Association may be performed before, during, and/or after a cardiac anomaly is detected. Association may include, for example, electronically receiving cardiac information and activity information associated with a common parameter, and storing the cardiac information, activity information, and temporal information in a manner that they may later be accessed and associated together based on the common parameter. In addition, in an embodiment, association may include receiving and/or retrieving anomaly information pertaining to a detected anomaly associated with the common parameter, and storing the cardiac information, activity information, and anomaly information in a manner that they may later be accessed and associated together based on the common parameter. In an embodiment, storing the anomaly information with the associated cardiac/activity information may serve to correlate the associated cardiac/activity information with the cardiac anomaly.

In an embodiment, information association element 412 is adapted to store associated cardiac/activity information in data storage apparatus 410 in the form of records within one or more tables and/or databases (e.g., a relational database). In an embodiment, information association element 412 enables the creation of multiple data records, each of which corresponds to a temporal parameter (e.g., a time instant, a start time, a stop time, a time period, a time duration and/or a counter value) and/or an event (e.g., a particular heartbeat and/or set of consecutive heartbeats). Each data record includes one or more items of associated cardiac/activity information associated with the temporal parameter and/or event, in an embodiment. As used herein, the term “data record” includes multiple fields of information which are stored together in memory or which are stored separately, but may be related together based on one or more common keys (e.g., common parameters). In other words, a data record may correspond to data stored within a single table or data stored within multiple tables that are related based on one or more common keys. A data record may include, for example, one or more temporal information fields, one or more cardiac information fields, and one or more activity information fields. In an embodiment, a data record may further include one or more additional fields of information that may be associated with the cardiac information, the activity information, and/or other types of information discussed herein. For example, Table 1 illustrates a database that includes multiple records having a particular set of fields:

TABLE 1

| | | Average | | | Average | |
|---|---|---|---|---|---|---|
| Start | Stop | Heart | RR | Measured | Caloric | |
| Time | Time | Rate | Interval | Activities | Consumption | Marker |
| 0 min | 5 min | 75 | 856 | lying | 88 | V-ARR |
| 5 min | 10 min | 77 | 769 | lying | 92 | — |
| 10 min | 15 min | 88 | 695 | lying; | 120 | — |
| | | | | standing; | | |
| | | | | walking | | |
| 15 min | 20 min | 96 | 625 | walking | 144 | S-ARR |

In Table 1, each record includes temporal information (i.e., start time and stop time), cardiac information (i.e., average heart rate and RR interval), and activity information (i.e., measured activities and average caloric consumption). In addition, each record includes anomaly information in the form of a marker field (e.g., “V-ARR” may indicate that a vagal arrhythmia was detected, and “S-ARR” may indicate that a sympathetic arrhythmia was detected). Table 1 is provided for example purposes only. In other embodiments, each record may include different temporal information, cardiac information, and/or activity information.

In an embodiment, upon a request from an external apparatus, or in response to some other trigger, associated cardiac/activity information (e.g., associated data records) may be output from data storage apparatus 410 and from system 400 via output device 413. In another embodiment, associated cardiac/activity information may spontaneously be output (e.g., continuously in the absence of a specific request or other trigger). When associated cardiac/activity information is included within records of a single table, then those records may be output. When associated cardiac/activity information is included within records of multiple tables, then the records from the multiple tables may be output and combined together by the external device or some other device. Output device 413 may include one or more ports (e.g., serial and/or parallel ports) and/or hardware adapted to output data over a wireless connection.

In an embodiment, system 400 may further include at least one patient input device (not illustrated), which is adapted to enable a patient to input information regarding events and/or other information (e.g., start/stop of work, stress level, meal/bed time, medication/intervention, and/or symptoms such as perceived heart palpitations, acute pectoral angina, shortness of breath or fatigue, for example). This patient-provided information may be associated (e.g., by information association element 412) with cardiac information and activity information, in an embodiment, based on a same common parameter used to associate the cardiac information and the activity information. For example, the patient provided information may be stored in a data record corresponding to the time that the patient input was received and/or the time that a symptom occurred, according to the patient. System 400 also may include other sensor apparatus (not illustrated), which may collect data that may be analyzed and/or associated (e.g., by information association element 412) with cardiac information and activity information based on the same common parameter. These other sensor apparatus may include, for example, any one or more vibration sensor, pressure sensor, change in pressure with time (dp/dt) sensor, magnetic sensor, respiration sensor, biomarker level sensor (e.g., cardiac troponin I and T, creatine phosphokinase, creatine phosphokinase myoglobin band, myoglobin, fatty acid binding protein, ischemia modified albumin, and/or lactic acid sensor), oxygen level sensor, carbon dioxide level sensor, glucose sensor, pH sensor, body temperature sensor, impedance sensor, and/or optical sensor, to name a few. Associated cardiac/activity information and/or an associated data record may include any one or more of the additional items of information described in this paragraph or elsewhere in the detailed description, along with other items of information.

In an embodiment, system 400 may be adapted simply to generate, store, and/or output associated cardiac/activity information. In other embodiments, system 400 may further be adapted to generate a response, under certain circumstances. In a particular embodiment, response generating element 416 may initiate a response upon detection of a cardiac anomaly. A response may be performed, for example, by one or more of cardiac stimulus element 418, external apparatus notification element 420, and/or a patient alert element 422, in various embodiments.

For example, response generating element 416 may initiate cardiac stimulus element 418 to generate a stimulus to the heart tissue (e.g., defibrillation stimulus, pacing stimulus, pacing rate adjustment, pacing characteristic adjustment, and/or pulse adjustment stimulus). In addition or alternatively, response generating element 416 may initiate external apparatus notification element 420 to communicate with an external apparatus (e.g., communication device 308 and/or computing device 310), which in turn may contact an emergency response system (e.g., a 911 system) or other entity (e.g., a contact person, doctor's office, etc.). In addition or alternatively, response generating element 416 may initiate patient alert element 422 to generate an audible, visual or mechanical alert, and/or to provide instructions to the patient via a display or speaker. Instructions may include, for example, instructions to reduce activity, to change posture or position, to take a blood sample and perform a biomarker test, and/or to seek medical attention.

Some or all of the blocks illustrated in FIG. 4 may be operationally coupled together as illustrated. In other embodiments, additional or different operational couplings may be present in the system. Further, some or all of the blocks illustrated in FIG. 4 may be incorporated into a single package (e.g., a single device). Alternatively, various blocks may be incorporated into different devices, and the information exchanged between the blocks may be exchanged between devices. Accordingly, the description set forth herein is not intended to be limited to any particular embodiment in which various apparatus and methods are included within and/or performed within particular devices.

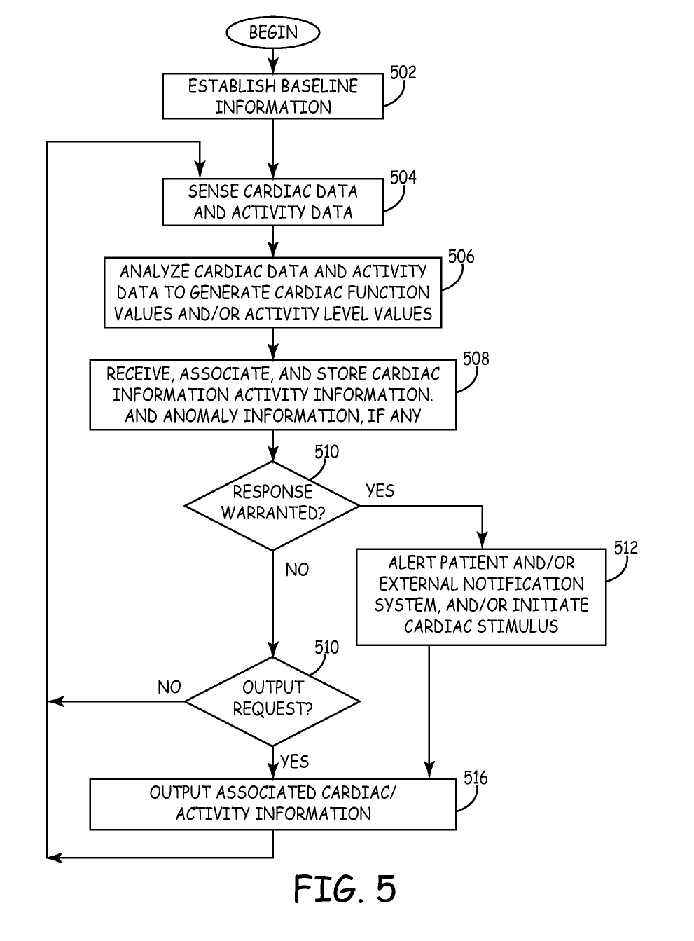

FIG. 5 illustrates a flowchart of a method for collecting and associating cardiac information and activity information, in accordance with an example embodiment. The method begins, in an embodiment, by establishing baseline information for the patient, in block 502. Establishing baseline information may include, for example, collecting (e.g., sensing), associating, and storing cardiac information and activity information, among other things, for a period of time associated with one or more cardiac cycles. The baseline information may include several sets of baseline information, including for example, cardiac information and activity information collected during periods of rest and/or activity, at different points in a respiration cycle, at various heart rates, at different body temperatures, and/or while the patient was positioned in various postural positions (e.g., sitting, supine and/or other). Desirably, the baseline information represents data generated while the patient was not encountering a cardiac anomaly (e.g., an arrhythmia). Establishing baseline information may be performed one time, occasionally, periodically, or continuously, in various embodiments. In another embodiment, baseline information may not be established prior to an observation period.

In block 504 cardiac data and activity data are sensed (e.g., collected). In an embodiment, data sensing may be performed continuously (e.g., in parallel with blocks 506-512). In an alternate embodiment, data sensing may be performed occasionally (e.g., periodically or in response to some triggering event). As described previously, cardiac data may include any one or more of a variety of types of cardiac data (e.g., ECG data, to name only one), which may be sensed by a variety of types of sensing apparatus. As also described previously, activity data may include any one or more of a variety of types of activity data (e.g., posture, to name only one), which may be sensed by a variety of types of sensing apparatus. In further embodiments, other types of cardiac data, activity data or other information may be sensed or collected, as well.

In block 506, portions of the sensed cardiac data and/or the sensed activity data may be analyzed to generate cardiac function values and/or activity level values, respectively. As mentioned previously, “cardiac information” includes cardiac data and/or cardiac function values, and “activity information” includes activity data and/or activity level values.

In addition, analysis of the cardiac information and the activity information may include determining whether a cardiac anomaly has occurred, the characteristics of the anomaly (e.g., the anomaly type and source), and/or whether the cardiac anomaly occurred during a period of rest or activity and/or during the day or at night (e.g., whether the anomaly was vagal or sympathetic). An anomaly may be detected and characterized, for example by analyzing cardiac information (and possibly activity information) to determine whether the information is characteristic of a cardiac anomaly and if so, what type. For example, in an embodiment, a portion of an ECG waveform may be analyzed (e.g., auto-correlated and/or compared to baseline ECG information), and the system may determine whether a correlation between the portion of the ECG waveform and the baseline ECG information indicates an arrhythmia or other cardiac anomaly. As a more particular example, the method may include analyzing the cardiac information to detect an arrhythmia during a portion of the observation period. This analysis may further include determining whether the arrhythmia is a bradycardia, a tachycardia or a fibrillation. In other embodiments, analysis may include, and/or determining whether a cardiac anomaly is atrial or ventricular, and/or vagal or sympathetic. In an embodiment, the system may generate anomaly information that indicates whether an anomaly has been detected, the type of anomaly (e.g., arrhythmia, bradycardia, tachycardia, fibrillation, ischemia, myocardial infarction, and so on), the source of the anomaly (e.g., atrial, ventricular, or other), and/or whether the anomaly is vagal or sympathetic.

In block 508, the cardiac information and the activity information are electronically received (e.g., from a cardiac sensor 402, cardiac data analyzer 404, activity sensor 406, activity data analyzer 408, and/or data storage apparatus 410, FIG. 4) and associated to generate associated cardiac/activity information. As discussed previously, the cardiac information and the activity information may be associated based on a common parameter (e.g., a common temporal parameter, a common event, and/or a common quality), in an embodiment. In additional embodiments, other information may be associated with the cardiac information and the activity information, as discussed previously. When a cardiac anomaly has been detected (e.g., in block 506), anomaly information corresponding to the anomaly may be associated with (e.g., stored with) the corresponding associated cardiac information and activity information. The associated cardiac/activity information may be stored in one or more data storage apparatus (e.g., data storage apparatus 410, FIG. 4). In an embodiment, substantially all of the associated cardiac/activity information that is generated may be stored. In an alternate embodiment, selected portions of the associated cardiac/activity information may be stored, such as those portions that correspond to a detected cardiac anomaly and/or those portions that correspond to a particular type of activity. In an embodiment, storage of the associated cardiac/activity information is essentially performed in real time, meaning that the information is stored as it is sensed, analyzed, and associated.

In block 510, a determination may be made whether the data analysis (e.g., the analysis performed in block 506) warrants a response. For example, when an anomaly is detected, a response may be warranted, although this may not always be the case. When no response is warranted, the method iterates as shown.

When a response is warranted, the system may initiate a response that is appropriate, in block 512. For example, as discussed previously, at least one response may be selected from a group of response types that includes generating a patient alert, contacting an external notification system, and initiating cardiac stimulus. As discussed previously, a patient alert may include an audible or vibratory alert, and/or an audible or displayed message. For example, when a detected cardiac anomaly is sympathetic, a patient alert may instruct the patient to reduce a level of the physical activity. Alternatively, a patient alert may instruct the patient to seek medical attention, take medication, or perform some other action. Contacting an external notification system may include sending a message to a patient communication device (e.g., device 308 FIG. 3) or a computing device (e.g., device 310 FIG. 3) indicating that an anomaly (e.g., atrial fibrillation, ventricular tachycardia, myocardial ischemia, myocardial infarction, or other anomaly) may be occurring. Initiating a cardiac stimulus may include generating a stimulus to the heart tissue (e.g., defibrillation stimulus, pacing stimulus, pacing rate adjustment, pacing characteristic adjustment, and/or pulse adjustment stimulus).

In block 514, a determination may be made whether a request for an output of associated cardiac/activity information has been made. For example, a request may be initiated by another, external apparatus (e.g., patient communication device 308 or communication device 310, FIG. 3, or another device) over a wired or wireless connection to download all or portions of the associated cardiac/activity information generated during a data collection period. Upon receipt of a request, the system may output the requested associated cardiac/activity information or some derivation thereof, in block 516. An embodiment may also include generating a recommended therapy, based on the cardiac information, the activity information, and/or the associated cardiac/activity information. Generation of the recommended therapy may be performed by the system and/or by the external device. The method may then iterate as shown.

Various processes are illustrated in series in FIG. 5. It is to be understood that some or all of the processes may be performed in parallel, and/or that the order of the processes may be altered while still achieving a substantially similar result. Therefore, variations in the ordering of the processes illustrated in FIG. 5 are intended to be included within the scope of the inventive subject matter.

Embodiments of the inventive subject matter may provide one or more technical, economic or other advantages over traditional systems. For example, embodiments of the inventive subject matter may provide systems, apparatus, and methods for more accurately and efficiently collecting and associating cardiac information (e.g., ECG data) with activity information, which may result in more rapid and accurate diagnoses of precipitating factors behind arrhythmias or other cardiac anomalies (e.g., myocardial ischemia, among other things). Use of the various embodiments may facilitate more accurate determinations of effective treatment strategies.

Embodiments of the inventive subject matter may be used in a number of diagnosis and/or treatment scenarios. For example, but not by way of limitation, embodiments may be incorporated into systems that provide continuous or occasional (e.g., periodic or event-initiated) heart monitoring for individuals at a high risk of having serious arrhythmia events (e.g., atrial fibrillation or ventricular tachycardia), myocardial infarction, and/or other cardiac anomalies. Additionally or alternatively, various embodiments may be used in emergent situations, such as in a hospital emergency room for patients that have symptoms cardiac distress. Various embodiments also or alternatively may be used in non-emergency situations in which a desire is present to collect and automatically associate cardiac information and activity information (e.g., in a clinic and/or doctor's office).

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the inventive subject matter. Rather, the foregoing detailed description will provide those skilled in the art with a convenient road map for implementing an embodiment of the inventive subject matter, it being understood that various changes may be made in the function and arrangement of elements described without departing from the scope of the inventive subject matter as set forth in the appended claims and their legal equivalents.

1. A method performed by an implantable cardiac apparatus, the method comprising the steps of:
generating cardiac information descriptive of cardiac functioning of a patient;
detecting a cardiac anomaly based on an analysis of the cardiac information;
generating activity information descriptive of physical activity of the patient during the cardiac anomaly;
associating the cardiac information and the activity information, during the cardiac anomaly, to generate associated cardiac/activity information; and
storing the associated cardiac/activity information.

2. The method of claim 1, further comprising:
storing anomaly information with the associated cardiac/activity information, which correlates the associated cardiac/activity information with the cardiac anomaly.

3. The method of claim 1, further comprising:
determining, based on the activity information, whether the cardiac anomaly is vagal or sympathetic; and
when the cardiac anomaly is sympathetic, generating a patient alert to instruct the patient to reduce a level of the physical activity.

4. The method of claim 1, further comprising:
determining, based on the activity information, whether the cardiac anomaly is vagal or sympathetic; and
storing anomaly information to indicate whether the cardiac anomaly is vagal or sympathetic.

5. The method of claim 1, further comprising:
determining, based on the cardiac information, whether the cardiac anomaly is atrial or ventricular; and
storing anomaly information to indicate whether the cardiac anomaly is atrial or ventricular.

6. The method of claim 1, further comprising:
determining, based on the cardiac information, whether the cardiac anomaly is tachycardia; and
when the cardiac anomaly is tachycardia, storing anomaly information to indicate that the cardiac anomaly is tachycardia.

7. The method of claim 1, further comprising:
determining, based on the cardiac information, whether the cardiac anomaly is a fibrillation; and
when the cardiac anomaly is fibrillation, storing anomaly information to indicate that the cardiac anomaly is fibrillation.

8. The method of claim 1, further comprising:
generating at least one recommended therapy based on the cardiac information and the activity information.

9. The method of claim 1, further comprising:
determining whether a response is warranted; and
when the response is warranted, initiating at least one response selected from a group of response types that includes generating a patient alert, contacting an external notification system, and initiating cardiac stimulus.

10. The method of claim 1, further comprising:
sensing at least one type of activity to generate the activity information, wherein the at least one type of activity sensed is selected from a group of activities that include limb movement, postures such as lying, reclining, sitting, standing, and leaning, posture transitions, gaits such as jumping, walking, running, going up stairs and going down stairs, biking, eating, and drinking.

11. The method of claim 1, further comprising:
outputting the associated cardiac/activity information to an external apparatus.

12. The method of claim 11, further comprising:
receiving a request for the associated cardiac/activity information from the external apparatus, wherein outputting the associated cardiac/activity information is performed in response to receiving the request.

13. An apparatus adapted to be implanted in a patient, the apparatus comprising:
at least one cardiac sensor adapted to generate cardiac information descriptive of cardiac functioning of the patient;
at least one activity sensor adapted to generate activity information indicating physical activity of the patient;
a processing element adapted to receive the cardiac information and to detect a cardiac anomaly based on an analysis of the cardiac information;
an information association element adapted to generate associated cardiac/activity information during the cardiac anomaly; and
at least one data storage apparatus adapted to store the associated cardiac/activity information.

14. The apparatus of claim 13, further comprising:
at least one output device adapted to output the associated cardiac/activity information to an external apparatus.

15. The apparatus of claim 13, further comprising:
at least one cardiac stimulus element adapted to stimulate heart tissue in response to detecting the cardiac anomaly.

16. The apparatus of claim 13, wherein the apparatus is selected from a group of apparatus that includes an implantable pulse generator, an implantable cardiac resynchronization therapy device, an implantable cardioverter defibrillator, and an implantable cardiac diagnostic and monitoring device.

17. The apparatus of claim 13, wherein the at least one cardiac sensor is selected from a group of sensors that includes an electrocardiogram (ECG) sensors, a heart sound sensor, a heart rate sensor, a heart rate variability sensor, and a heart wall motion sensor.

18. The apparatus of claim 13, wherein the at least one activity sensor is selected from a group of sensors that includes a movement sensor, a single axis accelerometer, a multiple-axis accelerometer, a body segment angle sensor, a strain gauge, and a pedometer.

19. A medical diagnostic system comprising:
an implantable cardiac apparatus that includes:
at least one cardiac sensor adapted to generate cardiac information descriptive of cardiac functioning of the patient,
at least one activity sensor adapted to generate activity information indicating physical activity of the patient,
a processing element adapted to receive the cardiac information and to detect a cardiac anomaly based on an analysis of the cardiac information,
an information association element adapted to generate associated cardiac/activity information during the cardiac anomaly,
at least one data storage apparatus adapted to store the associated cardiac/activity information, and
at least one output device adapted to output the associated cardiac/activity information; and
an external apparatus that includes
a receiver adapted to receive the associated cardiac/activity information, and
a display device adapted to display the associated cardiac/activity information.

20. The medical diagnostic system of claim 19, wherein the implantable cardiac apparatus is an apparatus selected from a group of apparatus that includes an implantable pulse generator, an implantable cardiac resynchronization therapy device, an implantable cardioverter defibrillator, and an implantable cardiac diagnostic and monitoring device.
